(19) United States
(12) Patent Application Publication
(10) Pub. No.: US 2025/0032159 A1
(43) Pub. Date: Jan. 30, 2025

(54) TPLO Plate Dual Vector Compression System and Method

(71) Applicant: DEPUY SYNTHES PRODUCTS, INC., Raynham, MA (US)

(72) Inventor: Timothy J. HORAN, Royersford, PA (US)

(21) Appl. No.: 18/357,700

(22) Filed: Jul. 24, 2023

(51) Int. Cl. A61B 17/80

(52) U.S. Cl. CPC A61B 17/8014; A61B 17/8061; A61B 2017/564; A61B 2017/681

(57) ABSTRACT

A plate includes Body and first and second distal holes. Body extends longitudinally from proximal to distal ends and defines via first and second surfaces. Body includes a proximal portion positioned over a cut and a distal portion extending along axis L. The first hole receives a first element and extends through a proximal end of the distal portion from the first to second surfaces. The first hole extends through the plate along axis B angled relative to axis L. The second hole extends through the distal portion distally of the first hole from the first to second surfaces. The second hole extends along axis L and includes a sloped surface along a distal portion thereof so that, when a second element is inserted into the second hole, contact between a head of the second element and the sloped surface generates a first distal translation of the plate relative to the second element. The first hole is configured such that, the first translation moves the plate relative to the first element so that the first element translates along axis B toward a proximal end of the first hole, moving the proximal portion distally and cranially.

### BACKGROUND

A Tibial Plateau Leveling Osteotomy (TPLO) is a surgical procedure for stabilizing a canine stifle joint (comparable to a human knee joint) after rupture of a cranial cruciate ligament (CCL). When the CCL is ruptured or torn, the animal's tibia slides forward with respect to its femur, making it difficult to walk and causing pain. To stabilize the joint, a curvilinear cut is made to the upper portion of the tibia. This cut portion of the tibia is then rotated to create a more level plane or surface on the top of the tibia, onto which the femur will rest. The cut and repositioned portion of the tibia is then secured to the lower portion of the tibia in a desired position and orientation using a TPLO plate.

TPLO plates are generally sized and shaped to extend along the cut portion of the tibia and the remaining portion of the tibia to facilitate healing of the tibia in its new configuration. In some cases, however, it may be difficult to properly seat the cut portion on the lower portion of the tibia or cut may not be compressed against the lower portion of the tibia to the extent desired. This may prevent the bone from healing as desired.

### SUMMARY OF THE INVENTION

The present disclosure relates to a plate for providing cranial and distal compression of an osteotomy cut. The plate includes a body extending longitudinally from a proximal end to a distal end and defined via a first surface which, in an operative configuration, faces away from a bone and a second surface which, in the operative configuration, faces toward the bone. The bhe body includes a proximal portion configured to be positioned over a cut and repositioned proximal segment of the bone and a distal portion extending along a longitudinal axis L and configured to be positioned over a distal segment of the bone.

The plate also includes a first distal hole extending through a proximal end of the distal portion of the body from the first surface to the second surface. The first distal hole extends through the plate along an axis B angled relative to the axis L. The first distal hole is configured to slidably receive a first bone fixation element.

In addition, the plate includes a second distal hole extending through the distal portion of the body distally of the first distal hole from the first surface to the second surface. The second distal hole extends along the axis L and including a sloped compression surface along a distal portion thereof configured so that, when a second bone fixation element is inserted into the second distal hole, contact between a head of the second bone fixation element and the sloped compression surface of the second distal hole generates a first distal translation of the plate relative to the second bone fixation element.

The first distal hole is configured such that, the first distal translation of the plate moves the plate relative to the first bone fixation element so that the first bone fixation element translates along the axis B toward a proximal end of the first distal hole, moving the proximal portion of the plate distally and cranially.

In an embodiment, an angle between the axis L and the axis B along which the first distal hole extends corresponds to a desired cranial displacement.

In an embodiment, the first distal hole is angled relative to the axis L so that a first end is proximal to a second end and the first end is separated transversely from the axis L on a caudal side of the axis L and the second end is separated transversely from the axis L on a cranial side of the axis L.

In an embodiment, the translation of the first bone fixation element along the axis B caused by the first distal translation of the plate rotates the plate about the second bone fixation element so that the proximal portion of the plate is moved cranially.

In an embodiment, the plate further includes a third distal hole extending through the distal portion of the body, between the first and second distal holes, from the first surface to the second surface and extending along the axis L, a distal portion of the third distal hole including a sloped compression surface.

In an embodiment, the third distal hole is configured as a combi-hole including a proximal portion configured as a locking hole and a distal portion configured as a dynamic compression hole, the proximal and distal portions of the combi-hole being open to and in communication with one another.

In an embodiment, when the third distal hole is configured so that, as a third bone fixation element is inserted into the third distal hole the sloped compression surface of the third distal hole provides a second distal translation of the plate relative to the third bone fixation element, and wherein the first distal hole is configured such that, the second distal translation of the plate moves the plate relative to the first bone fixation element so that the plate moves relative to the first bone fixation element so that the first bone fixation element moves along the axis B toward a first end of the first distal hole, moving the proximal portion of the plate further distally and cranially.

In an embodiment, the translation of the plate relative to the first bone fixation element along the axis B caused by the second distal translation of the plate causes the plate to rotate about the third bone fixation element so that the proximal portion of the plate moves cranially.

In an embodiment, the plate is configured so that rotation of the plate about the third bone fixation element as the third bone fixation element is inserted into the third distal hole moves the second bone fixation element laterally relative to the axis L, and wherein the second distal hole comprises a relief portion sized, shaped and positioned to accommodate the lateral motion so that the second bone fixation element does not contact an inner surface of the second distal hole and prevent achievement of a desired degree of compression.

In addition, the present disclosure relates to a method for providing cranial and distal compression of an osteotomy cut. The method includes positioning a bone plate in a desired initial position with a first surface of the bone plate facing away from a tibia and a second surface thereof facing the tibia so that a distal portion of the bone plate extends over a distal tibial segment and a proximal portion of the bone plate extends over a proximal tibial segment that has been cut away from the proximal tibial segment, rotated and seated within a recess formed in the distal tibial segment when the proximal tibia segment was cut away; coupling the proximal portion of the bone plate to the proximal tibial segment; inserting a first distal bone fixation element into the distal tibial segment of the tibia via a first distal hole, the first distal hole extending along an axis B angled relative to a longitudinal axis L of a distal portion of the bone plate; and inserting a second distal bone fixation element into the distal tibial segment via a second distal hole extending through the distal portion of the bone plate distally of the first distal hole so that a head portion of the second distal bone fixation element slides along a sloped compression surface forming an edge of a distal portion of the second distal hole to move the bone plate distally relative to the second distal bone fixation element pulling the proximal portion of the bone plate and the proximal tibial segment distally to provide a first distal compression between the proximal tibial segment and the distal tibial segment, the bone plate translating relative to the first distal bone fixation element so that the first distal bone fixation element translates proximally along the axis B during the first distal compression rotating the bone plate about the second distal bone fixation element so that the proximal portion of the bone plate moves cranially to provide a first cranial compression of the proximal tibial segment against the distal tibial segment.

In an embodiment, an angle between the axis L and the axis B corresponds to a desired cranial displacement and the first distal hole is angled so that a first end is proximal to a second end and the first end is separated transversely from the axis L on a caudal side of the axis L and the second end is separated transversely from the axis L on a cranial side of the axis L.

In an embodiment, the translation of the first distal bone fixation element along the axis B caused by the first distal translation of the bone plate rotates the bone plate about the second distal bone fixation element to move the proximal portion of the bone plate cranially.

In an embodiment, a third distal hole extends through the distal portion of the bone plate, between the first and second distal holes, from the first surface to the second surface, the third distal hole extending parallel to the axis L, a distal portion of the third distal hole including a sloped compression surface.

In an embodiment, the method further includes inserting a third bone fixation element into the third distal hole so that the sloped compression surface of the third distal hole provides a second distal translation of the bone plate relative to the third bone fixation element and a second cranial compression of the proximal tibial segment against the distal tibial segment.

In an embodiment, the rotation of the bone plate about the third bone fixation element moves the bone plate relative to the second distal bone fixation element so that the second distal bone fixation element moves laterally relative to the second distal hole, and wherein the second distal hole comprises a relief portion sized, shaped and positioned to accommodate the lateral motion of the second distal bone fixation element so that the second distal bone fixation element does not contact an inner surface of the second distal hole and prevent a desired degree of compression.

### BRIEF DESCRIPTION

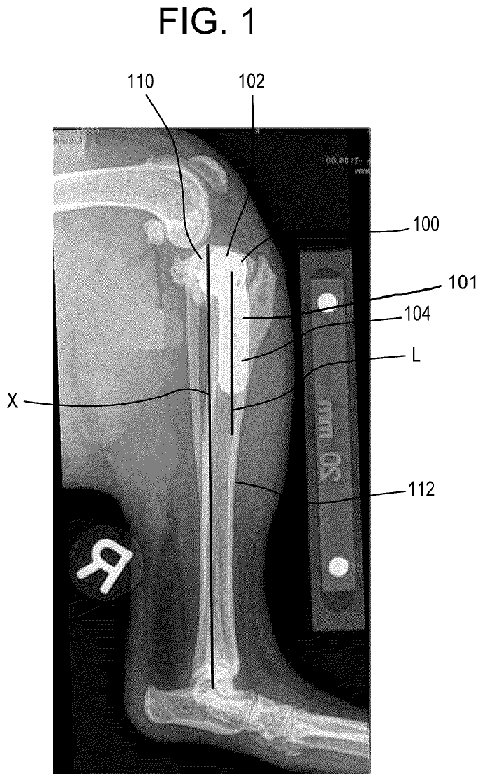

FIG. 1 shows a TPLO bone plate according to one example.

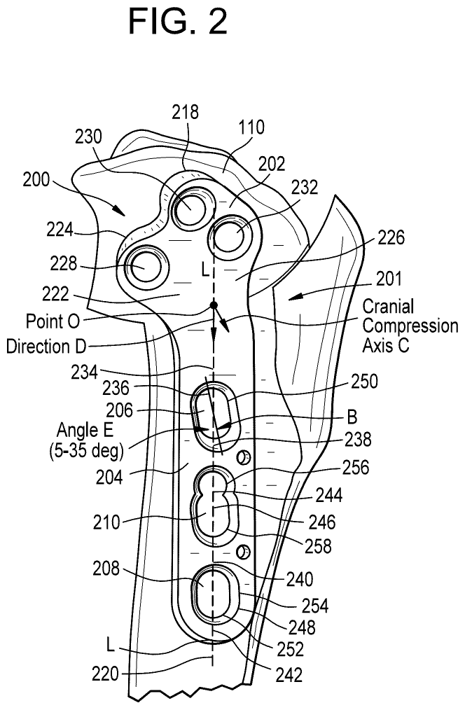

FIG. 2 shows a plan view of a TPLO bone plate according to an exemplary embodiment of the present disclosure.

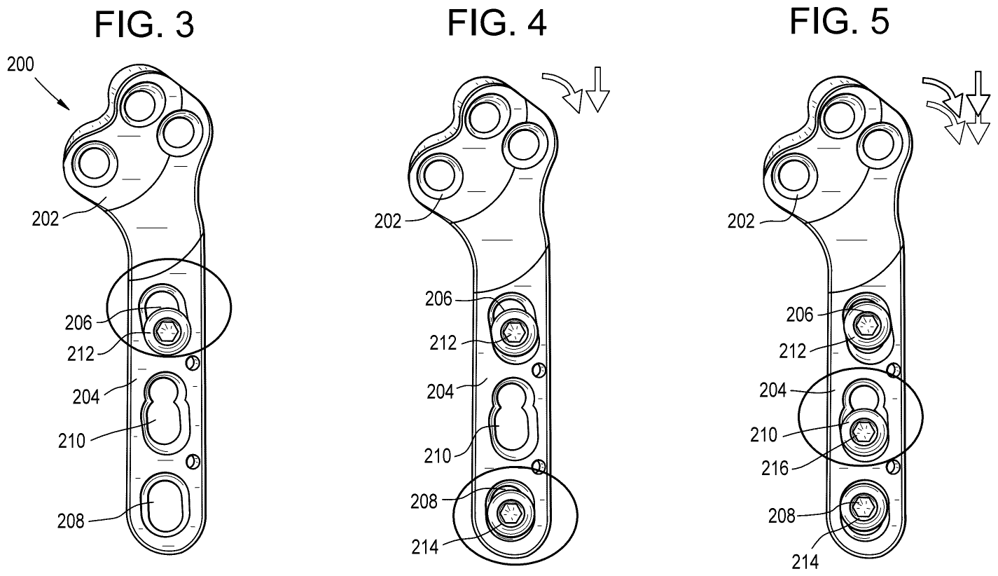

FIG. 3 shows the bone plate of FIG. 2 with a first bone fixation element inserted loosely into a first hole.

FIG. 4 shows the bone plate of FIG. 2 with a second bone fixation element inserted into a second hole.

FIG. 5 shows the bone plate of FIG. 2 with a third bone fixation element inserted into a third hole.

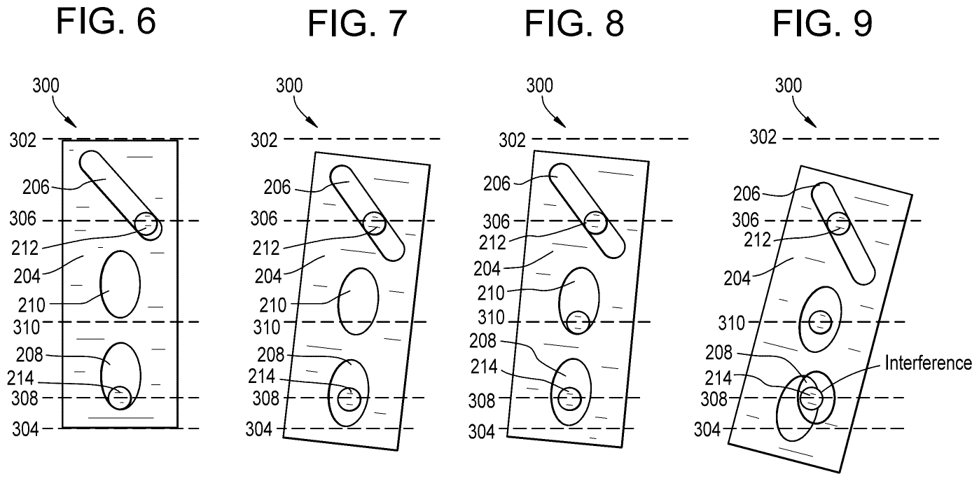

FIG. 6 shows a distal portion of a TPLO bone plate according to an exemplary embodiment with a first bone fixation element inserted loosely in a first hole and a second bone fixation element inserted in a second hole but not yet compressed.

FIG. 7 shows the first compression of the plate of FIG. 6.

FIG. 8 shows a third bone fixation element inserted in a third hole of the plate of FIG. 6, but not yet compressed.

FIG. 9 shows the second compression of the plate of FIG. 6.

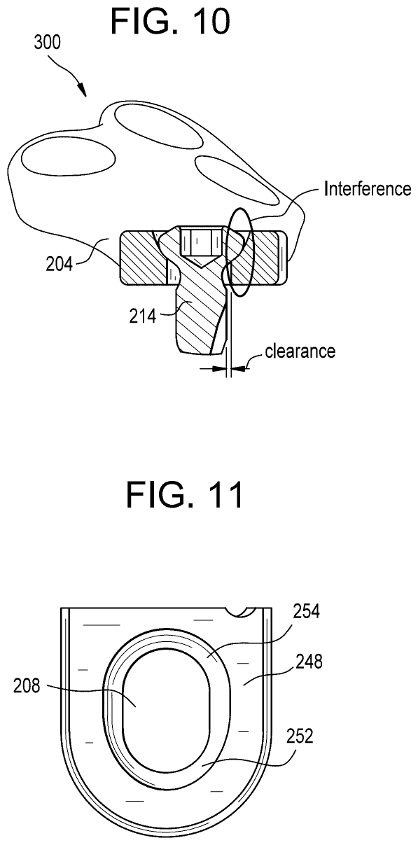

FIG. 10 shows the interference between the second bone fixation element and the bone plate of FIG. 6 after the second compression.

FIG. 11 shows the detailed design of the second hole of the bone plate of FIG. 2 including a relief portion to accommodate the distal/cranial plate shift while avoiding interference with the head of the second bone fixation element.

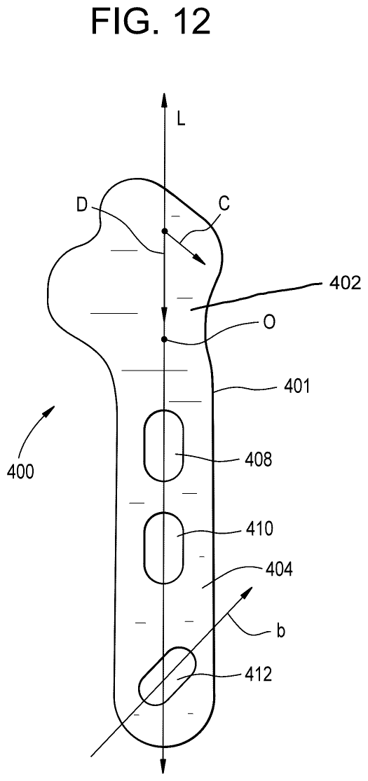

FIG. 12 shows a plan view of a bone plate according to a further embodiment.

### DETAILED DESCRIPTION

The present disclosure may be further understood with reference to the following description and the appended drawings, wherein like elements are referred to with the same reference numerals. The present disclosure relates to a Tibial Plateau Leveling Osteotomy (TPLO) plate and, in particular, relates to a TPLO plate configured to provide both cranial (i.e., transverse) and distal (i.e., axial) compression during a TPLO procedure. Exemplary embodiments of the present disclosure describe a TPLO plate comprising a proximal portion configured to be positioned over a cut and repositioned upper portion (e.g., proximal portion) of a tibia, and a distal portion configured to be positioned along a lower portion (e.g., distal portion) of the tibia.

The distal portion of the plate according to an exemplary embodiment includes three holes positioned along the length of the distal portion so that bone fixation members inserted into these holes work in concert to provide both cranial and distal compression of the interface between the cut and repositioned portion of the tibia and the lower portion of the tibia. Those skilled in the art will recognize that additional holes may be provided in any suitable arrangement if desired. The first hole on the distal portion of the plate according to this embodiment is elongated along an axis angled with respect to a longitudinal axis of the distal portion of the plate. The second hole is an elongated compression hole that extends along the longitudinal axis; the second hole is located distally of the first hole.

Thus, when a first bone fixation element is inserted through the first hole into the bone without being completely tightened (e.g., so that the bone plate can slide relative to the first bone fixation element only along the axis of the first hole) and a second bone fixation element is inserted into the bone via the second hole, tightening of the second bone fixation element forces the plate to slide distally relative to the second bone fixation element (e.g., along the longitudinal axis) and, due to the angulation of the first hole relative to the longitudinal axis, rotates the plate about the second bone fixation element. The angulation of the axis of the first hole with respect to the longitudinal axis is selected so that the rotation of the plate as the first bone fixation element traverses the first hole moves the proximal portion of the plate (and the cut and rotated section of bone coupled thereto) relative to the lower portion of the tibia to provide a desired level of cranial compression at the interface between the cut portion of bone and the lower tibia while movement of the plate distally relative to the first and second bone fixation elements moves the proximal portion of the plate distally to simultaneously apply a desired level of distal compression across the osteotomy cut.

The distal portion of the plate further includes a third hole extending therethrough, positioned distally of the first hole and proximally of the second hole. The third hole is configured so that, when engaged via a third bone fixation element inserted therethrough, the plate is moved further distally relative to the tibia and the plate further rotates so that the position of the first bone fixation element within the first hole is increasingly proximal. Tightening the third bone screw in the third hole generates a second cranial compression across the osteotomy cut in addition to the first cranial compression as well as a second distal compression across the osteotomy cut.

In an embodiment, the second hole includes a relief portion that is sized and shaped to accommodate the lateral shift of the plate relative to the second bone fixation element to eliminate interference between a screw head of the second bone fixation element and the bone plate that would otherwise prevent the desired movement of the bone plate relative to the second bone fixation element.

It will be understood by those of skill in the art that although the TPLO plates of the present embodiments are described with respect to a canine CCL, the TPLO plate of the present disclosure may also be used to treat the tibias of other quadrupeds such as, for example, felines, bovines, equines, etc. It will also be understood by those of skill in the art that the terms proximal, distal, caudal, and cranial, as used herein, are anatomical directional terms for an animal such as, for example, a canine and are employed in a manner consistent with their standard anatomical meanings. In particular, the term proximal refers to a direction toward a center of the body while distal refers to a direction away from the center of the body while cranial refers to a direction toward a head of the animal and caudal refers to a direction away from the head.

FIG. 1 shows a TPLO bone plate 100 according to one example implanted in a TPLO operation. As indicated above, the bone plate 100 is configured to secure proximal and distal segments of a tibia which have been separated from one another through a curvilinear osteotomy during a TPLO procedure as would be understood by those skilled in the art. As indicated above, in a TPLO procedure a proximal segment of the tibia is cut away from the rest of the tibia, rotated relative to the tibia to a new position and orientation selected to enhance the stability of, for example, a canine stifle joint (e.g., after injury to a cranial cruciate ligament (CCL)), and secured to the distal segment in this desired position—e.g., via a bone plate coupled to the cut segment of the tibia and the distal segment of the tibia.

In this example, the bone plate 100 is capable of distal compression only. The bone plate 100 of this example is described in brief relative to the bone plates described below according to these exemplary embodiments. The bone plate 100 comprises a body 101 including a proximal portion 102 sized and shaped to be positioned over and coupled to a first segment 110 of bone, e.g., the cut and repositioned proximal segment of the tibia, and a distal portion 104 sized and shaped to extend along and be coupled to a second segment 112 of bone, e.g., the distal segment of the tibia, so that the bone plate 100 fixes the position of the first segment 110 relative to the second segment 112. The distal portion 104 extends along a longitudinal axis L (parallel to the longitudinal axis of the bone X) and includes a number of holes (e.g., three holes) extending therethrough. When bone fixation elements are inserted in the holes, a desired level of distal compression of the osteotomy cut is provided at the interface between the first and second segments 110, 112 of bone. In this example, the interface is at a caudal position and a cranial gap exists in the cranial direction.

FIGS. 2-5 show a TPLO bone plate 200 according to an exemplary embodiment of the present disclosure. The bone plate 200 comprises a body 201 including a proximal portion 202 sized and shaped to be positioned over and coupled to a first segment of bone and a distal portion 204 sized and shaped to extend along and be coupled to the second segment of bone. The distal portion 204 extends along a longitudinal axis L and includes a number of holes, e.g., three holes, extending therethrough and the distal compression is applied in the direction D along the axis L. The holes of the distal portion 204 are configured so that, when bone fixation elements are inserted in the holes, desired levels of distal and cranial compression of the osteotomy cut are provided at the interface between the first and second segments 110, 112 of bone.

Those skilled in the art will understand that the amount of cranial compression will depend on basic geometric relations between the angle at which the axis along which an angled hole extends and the distances between (1) the screw about which the plate is rotating (e.g., screw) and a point (O) on the bone plate that is configured to overlie the osteotomy; and (2) the distance between the angled hole and the point O. Thus, the same amount of cranial compression (for a given distal compression) may be obtained by a bone plate in which the angled hole is distal of the hole including the screw about which the plate is rotating (See FIG. 12). As described below, in such a plate, the angled hole would be oriented in an opposite direction relative to the longitudinal axis L of the plate and would have an angle increased to reflect the larger distance from the angled hole to the point O as compared to the distance from the point O to the screw about which the plate is rotating. Thus, those skilled in the art will understand that the desired amount of cranial compression may be achieved by selecting the location (relative to the point O) and angle of the angled hole and the position (relative to the point O) of the hole configured to receive the screw about which the plate will rotate.

Specifically, cranial compression is applied along an axis C as the bone plate 200 rotates cranially to eliminate a gap between the first and second segments 110, 112 of bone and the rounded and cut portion of the first segment 110 is fits snugly into the rounded and cut portion of the second segment 112.

In an exemplary embodiment, the distal portion 204 of the bone plate 200 includes three holes—a first hole 206, a second hole 208, and a third hole 210—extending therethrough. Those skilled in the art will understand that additional holes may be added as desired so long as they do not impede the functioning of the bone plate 200 with respect to the distal and cranial compression as will be described below. The first hole 206 is elongated along an axis B angled with respect to the longitudinal axis L of the distal portion 204 of the bone plate 200.

As would be understood by those skilled in the art, the first hole 206 includes a stem receiving portion adjacent to a bone facing surface of the bone plate 200 that has a width selected to receive a stem of a bone fixation element so that the bone plate 200 can slide relative to the bone fixation element while a head of the bone fixation element is too wide to pass through this stem receiving portion of the first hole 206. Thus, the bone plate 200 can move relative to a bone fixation element implanted into the bone through the first hole 206 only along the axis B.

Each of the bone fixation element receiving holes of the bone plate 200 is similarly configured with a stem receiving portion adjacent to the bone facing surface of the bone plate 200 and a head receiving portion extending from the stem receiving portion to an outer surface of the bone plate 200 which, when the bone plate 200 is positioned on a target portion of bone as desired, faces away from the bone. The second hole 208 is elongated along the longitudinal axis L and is distal of the first hole 206. As will be described in further detail below, the first hole 206 and the second hole 208 are configured to work together so that, when first and second bone fixation elements 212, 214 are inserted therein, respectively, in a desired order, desired levels of distal and cranial compression are provided across the osteotomy cut at the interface between the cut and repositioned portion of the bone and the distal segment of the tibia.

The first hole 206, the second hole 208 and the third hole 210 are configured (positioned, sized and oriented) to work together so that, when a third bone fixation element 216 is subsequently inserted into the third hole 210, a desired amount of additional distal and cranial compression is provided at the interface between the cut and repositioned portion of the bone and the distal segment of the tibia. The amounts of cranial and distal compression are selected to ensure that the cut and repositioned proximal segment of the tibia is seated within a recessed portion of the distal segment formed via the curvilinear cut to optimize healing of the bone.

As shown in FIG. 2, the bone plate 200 includes a body 201 extending longitudinally from a proximal end 218 to a distal end 220. The body 201 is defined via a first surface 222 which, in an operative configuration, faces away from a bone (e.g., the tibia), and a second surface 224 which, in the operative configuration, faces toward the bone. The body 201 includes the proximal portion 202 and the distal portion 204, which are connected to one another via a neck portion 226 so that, when in the operative configuration, the proximal portion 202 is positioned over and coupled to the proximal segment of the tibia, as desired, and the distal portion 204 extends over and is coupled to the distal segment of the tibia with the neck portion 226 extending across the interface between the cut-away portion of the proximal tibia and the distal tibia at the curvilinear osteotomy cut.

The distal portion 204 extends distally of the proximal portion 202 along the longitudinal axis L. According to an exemplary embodiment, the neck portion 226 is curved so that the proximal portion 202 is angled with respect to the longitudinal axis L along which the distal portion 204 extends. For example, the proximal portion 202 may be angled in a caudal direction relative to the longitudinal axis L although, as would be understood by those of skill in the art, the angle may be selected in any manner desired to conform to the geometry of the cut-away and rotated portion of the tibia and its desired final position relative to the distal tibia (or any other bone segment involved) in any given procedure.

As will be understood by those of skill in the art, the proximal portion 202 is, in this embodiment, preferably constructed, sized, shaped, and contoured to conform to the shape and orientation of the cut-away proximal segment of the tibia when the cut-away segment is in a desired position relative to the distal tibia and the distal portion 204 is in a desired position on the distal tibia. In particular, the second surface 224 of this embodiment is specifically contoured so that, when the proximal portion 202 is positioned over the proximal segment of the tibia, the second surface 224 extends along an exterior surface of the cut and rotated proximal segment of the tibia, in contact therewith.

According to an exemplary embodiment, the proximal portion 202 includes at least three holes—a first hole 228, a second hole 230, and a third hole 232—each of which extends through the proximal portion 202 along a central axis, from the first surface 222 to the second surface 224. The first, second and third holes 228, 230, 232 may be positioned adjacent to an edge extending along the proximal end 218 of the bone plate 200. Each of the first, second and third holes 228, 230, 232 of the proximal portion 202 of this embodiment is configured to receive therein a bone fixation element such as, for example, a locking screw to fix the proximal portion 202 of the bone plate 200 relative to the cut and rotated proximal segment of the tibia.

In an exemplary embodiment, the first hole 228 is positioned on the bone plate 200 and oriented so that, when the bone plate 200 is positioned on a tibia in a desired position, the first hole 228 is positioned to receive a bone fixation element through a caudal portion of the resected proximal segment of the tibia, while the second hole 230 is positioned and oriented on the bone plate 200 to receive a bone fixation element through a proximal portion of the resected proximal segment of the tibia, and the third hole 232 is positioned and oriented on the bone plate 200 to receive a bone fixation element through a cranial portion of the resected proximal segment of the tibia. As would be understood by those skilled in the art, central axes of each of the first, second and third holes 228, 230, 232 may be optionally angled to direct bone fixation elements inserted therealong into a desired portion of the proximal segment of the bone (e.g., a central mass of the resected portion of the proximal tibia) and to minimize the possibility of any of these bone fixation elements extending out of the proximal segment of the bone into the knee joint.

Furthermore, any or all of the first, second and third holes 228, 230, 232 of the proximal portion 202 may be configured as locking holes, including a threading formed in a head receiving portion there of configured to engage corresponding threading on the head of a bone fixation element to be inserted therein. Thus, bone fixation elements inserted therein may be locking screws including corresponding threading along a head portion thereof. It will be understood by those of skill in the art, however, that the first, second and third holes 228, 230, 232 of the proximal portion 202 of the bone plate 200 may have any of a variety of configurations so long as bone fixation elements (e.g., bone screws or pins) are insertable therethrough to be inserted into a desired portion of the bone.

The distal portion 204 of the bone plate 200 is contoured to extend, when in the desired position, along the distal segment of the tibia. In particular, in the operative configuration, the second surface 224 extends along the distal segment, in contact therewith. As described above, the distal portion 204 of this embodiment also includes three holes extending therethrough—the first hole 206, the second hole 208 and the third hole 210. The first hole 206, the second hole 208, and the third hole 210 work in concert to provide desired levels of cranial and distal compression to the osteotomy cut of the tibia.

The first hole 206 is positioned adjacent to a proximal end 234 of the distal portion 204, adjacent to the neck portion 226. The first hole 206 extends through the bone plate 200 from the first surface 222 to the second surface 224 and is configured to receive the first bone fixation element 212 therein. The first hole 206 extends from a first end 236 to a second end 238 along the axis B, which is angled with respect to the longitudinal axis L by an angle E. The first end 236 is proximal to and caudal of the second end 238. The first hole 206 is angled so that the first end 236 (proximal end) is separated transversely from the longitudinal axis L on the caudal side of the axis L and the second end 238 (distal end) is separated transversely from the axis L on the cranial side of the axis L.

FIG. 3 shows the bone plate 200 with the first bone fixation element 212 inserted loosely into the first hole 206 (i.e., with the stem of the first bone fixation element 212 passing through the first hole 206 into the bone while the head of the first bone fixation element 212 is not yet firmly seated against the bone plate 200). Initially, the first bone fixation element 212 is positioned adjacent to the second end 238 of the first hole 206 and, after the element 212 has been inserted loosely into the bone as desired, the second bone fixation element 214 is inserted through the second hole 208 as will be described in greater detail below.

As would be understood by those skilled in the art, the second hole 208 is formed as a compression hole with a head receiving portion of the second hole 208 configured so that, as the second bone fixation element 214 is inserted further into the second hole 208 and the head of the second bone fixation element 214 is driven into contact with the head receiving portion of the second hole 208, the bone plate 200 translates distally relative to the first and second bone fixation elements 212, 214, respectively. The angle of the axis B is selected so that, as the bone plate 200 moves distally relative to the first and second bone fixation elements 212, 214, respectively, the bone plate 200 (and the proximal portion of the tibia attached thereto) rotates so that the proximal portion 202 of the bone plate 200 moves distally and cranially to press the proximal portion of the tibia against the surface of the osteotomy cut (i.e., to apply cranial and distal compression across the osteotomy cut), as will be described in further detail below.

Inner surfaces 250 defining the first hole 206 through the distal portion 204 are, in this embodiment, configured to correspond to a size of a head portion of the first bone fixation element 212. For example, the inner surfaces 250 of this embodiment taper from the first surface 222 toward the second surface 224 and/or include a curvature corresponding to a curvature of an underside the head portion (e.g., a surface of the head portion which is configured to engage the surfaces of the bone plate 200) so that upon insertion of the first bone fixation element 212 thereinto, the head portion of the first bone fixation element 212 is slidable along the axis B, as will be described in further detail below.

In addition, the first hole 206 of this embodiment is configured with a length selected so that, when the first bone fixation element 212 is initially positioned as desired (e.g., adjacent to a distal end of the first hole 206), the head portion of the first bone fixation element 212 is, after the application of a total desired distal compression, seated along the inner surfaces 250 flush with the first surface 222 of the bone plate 200, as will be understood by those of skill in the art. In an exemplary embodiment, the first bone fixation element 212 is a standard cortex screw.

In an exemplary embodiment, the angle E between the longitudinal axis L and the axis B may range from between approximately 5 degrees to 35 degrees. The first hole 206, however, may have any of a variety of angulations relative to the longitudinal axis L depending on the positions of the first and second holes 206, 208 along the axis L and a desired amount of cranial and distal compression.

As would be understood by those skilled in the art, the angle E is selected based on a desired cranial compression to be provided by the bone plate 200 for a given amount of distal compression. That is, those skilled in the art will understand the geometric relationships that allow the bone plate 200 to be designed to provide a desired amount of rotation of the proximal portion of the bone plate 200 (cranial compression) for a given amount of distal compression.

The second hole 208 extends through the distal portion 204, distally of the first hole 206, and is configured to receive the second bone fixation element 214 therein. In an exemplary embodiment, the second hole 208 extends through the distal portion 204 proximate the distal end 220 and has a length extending in longitudinal alignment with the longitudinal axis L. The second hole 208 in this embodiment is configured as a dynamic compression hole configured to provide distal compression across the osteotomy cut. For example, the second hole 208 of this embodiment extends through the distal portion 204 from the first surface 222 to the second surface 224 and is elongated along the longitudinal axis L. The second hole 208 extends from a first end 240 (proximal end) to a second end 242 (distal end), which facilitates translational movement of second hole 208 relative to the second bone fixation element 214 that is received therein so that the second bone fixation element 214 traverses from a position adjacent to the second end 242 to a position between the first end 240 and the second end 242 as a first distal and cranial compression is applied to the tibia.

FIG. 4 shows the bone plate 200 with the second bone fixation element 214 inserted into the bone via the second hole 208. The bone plate 200 moves distally and rotates so that the proximal portion 202 moves cranially as the second bone fixation element 214 is tightened into the second hole 208. The second end 242 of the second hole 208 includes a sloped compression surface 252 inclined at a curve/angle selected so that, as an underside of a head portion of the second bone fixation element 214 is pressed thereagainst (e.g., as the third bone fixation element 216 is inserted gradually further into the bone and further through the bone plate 100, to be described in greater detail below), the sloped compression surface 252 slides along the head portion of the second bone fixation element 214.

In particular, as the second bone fixation element 214 is driven more deeply into the bone, the head portion of the second bone fixation element 214 slides along the sloped compression surface 252 moving the bone plate 200 distally relative to the second bone fixation element 214. This applies distal compression across the osteotomy—i.e., the proximal segment of the tibia is pulled distally against the distal segment of the tibia as the proximal portion 202 of the bone plate 200 coupled to the cut-away proximal segment of the tibia, is drawn distally by the movement of the distal portion of the bone plate 200.

According to an exemplary embodiment, the first end 240 of the second hole 208 is defined via a surface 254 sized, shaped, and configured to correspond to the size and shape of an underside of the second bone fixation element 214 which, in one embodiment, is a cortex screw. In an exemplary embodiment, the surface 254 is tapered and/or curved from the first surface 222 toward the second surface 224. More particularly, in one example, the surface 254 is configured as a spherical relief configured to seat the head portion of the second bone fixation element 214 therein so that, when fully inserted into the bone, the head portion of the second bone fixation element 214 is substantially flush with the first surface 222 of the body 201 of the bone plate 200 as would be understood by those skilled in the art. It will also be understood by those of skill in the art, however, that the head portion of the second bone fixation element 214 is not required to be finally seated within the spherical relief as the bone plate 200 moves so that the second bone fixation element 214 passes from the position adjacent to the second end 242 (distal end) toward the first end 240 (proximal end) via a distance corresponding to a desired axial compression.

As discussed above, the first hole 206 and the second hole 208 work in concert so that, when the first bone fixation element 212 and the second bone fixation element 214 are inserted into respective ones of the first and second holes 206, 208, distal and cranial compression is applied to the osteotomy cut. In particular, the first bone fixation element 212 is inserted into the first hole 206 adjacent to the second end 238 (distal end) of the first hole 206, e.g., loosely, and the second bone fixation element 214 is subsequently inserted into the second hole 208 adjacent to the second end 242 (distal end) of the second hole 208 so that, when driven into the bone, the head portion of the second bone fixation element 214 engages the sloped compression surface 252 so that the bone plate 200 moves distally relative to the second bone fixation element 214.

As this first compression is applied, the bone plate 200 translates relative to the first bone fixation element 212 along the axis B rotating the proximal portion 202 of the bone plate 200 cranially about the second bone fixation element 214 (clockwise, in FIG. 5). Thus, the proximal portion 202 of the bone plate 200, which is coupled to the cut proximal segment of the tibia, moves in a cranial direction, applying a cranial compression across the osteotomy cut. Thus, this portion of the first distal compression simultaneously applies the cranial compression in a known ratio based on the geometric relations of the locations of the plate holes relative to the point O and the angle of the axis B as described above.

The third hole 210 extends through the distal portion 204 and extends between the first and second holes 206, 208 (i.e., distal of the first hole 206 and proximal of the second hole 208), and is configured to receive the third bone fixation element 216 therein. In an exemplary embodiment, the third hole 210 extends through the body 201 from the first surface 222 to the second surface 224 and has a length extending along the longitudinal axis L, substantially in alignment with the second hole 208. In one exemplary embodiment, the third hole 210 is configured as a combi-hole including a proximal portion 244 configured to provide locking fixation of the bone plate 200 relative to the bone and a distal portion 246 configured to provide dynamic compression as would be understood by those skilled in the art. The proximal and distal portions 244, 246 of such a combi-hole are open to and in communication with one another.

The proximal portion 244 in this embodiment is configured as a locking hole extending through the first surface 222 to the second surface 224 along a central axis, which extends perpendicular relative to the longitudinal axis L. In one embodiment, the central axis of the proximal portion 244 extends substantially perpendicular to the longitudinal axis L. It will be understood by those of skill in the art, however, that the central axis of the proximal portion 244 may be non-perpendicularly angled relative to the central axis of the proximal portion 244 so that a bone fixation element inserted therealong may be inserted into a desired portion of a bone offset from the portion immediately beneath the axis L. A surface 256 defining the proximal portion 244 may include a threading extending therealong for engaging a corresponding threading along a head of, for example a locking screw, to provide a locking fixation between the plate and the bone.

Similarly to the second hole 208, the distal portion 246 in this embodiment includes a sloped compression surface 258 inclined at a curve/angle selected so that, as an underside of a head portion of the third bone fixation element 216 is pressed thereagainst (e.g., as the third bone fixation element 216 is inserted gradually further into the bone and further through the distal portion 246), the sloped compression surface 258 slides along the head portion of the third bone fixation element 216. As the third bone fixation element 216 is driven further into the bone, the head portion of the third bone fixation element 216 slides along the sloped compression surface 258 moving the bone plate 200 distally relative to the third bone fixation element 216. In this embodiment, the third bone fixation element 216 is a standard cortex screw.

FIG. 5 shows the bone plate 200 with the third bone fixation element 216 inserted into the third hole 210. The bone plate 200 moves further distally and rotates so that the proximal portion 202 moves further cranially as the third bone fixation element 216 is tightened. As discussed above, the first hole 206 and the third hole 210 work in concert so that, when the third bone fixation element 216 is subsequently inserted into the distal portion 246 of the third hole 210, the head portion of the third bone fixation element 216 slides along the sloped compression surface 258 providing a second distal compression across the osteotomy cut, drawing the cut proximal segment of the tibia which is coupled to the proximal portion 202 of the plate, further distally against the distal segment of the tibia.

As the bone plate 200 is moved distally relative to the third bone fixation element 216, the bone plate 200 translates relative to the first bone fixation element 212 so that the first bone fixation element moves relative to the first hole 206 toward the first end 236 (proximal end), causing the proximal portion 202 of the bone plate 200 to rotate further cranially about the second and third bone fixation elements 214, 216 (clockwise, in FIG. 5). As indicated above, movement of the proximal portion 202 of the bone plate 200, which is coupled to the cut proximal segment of the tibia, in a cranial direction, applies cranial compression across the osteotomy cut. Thus, this portion of the second distal compression simultaneously applies the cranial compression along with the desired distal compression.

Referring back to the second hole 208, in an embodiment, the second hole 208 includes a relief portion 248 sized shaped and positioned to accommodate the distal/cranial plate shift (i.e., to avoid interference between the head of the second bone fixation element 214 and the edges of the second hole 208) during the second distal compression. As would be understood by those skilled in the art, during the second distal compression, the bone plate 200 rotates cranially about the third bone fixation element 216 such that, without a relief portion 248 cut into the second hole 208, the second bone fixation element 214 might contact the inner surface 250 of the second hole 208 and prevent the bone plate 200 from achieving the desired compression.

FIGS. 6-11 show an exemplary scenario in which the compression of a TPLO bone plate 300 might cause interference. Except as detailed below, the bone plate 300 according to this example is substantially similar to the bone plate 200, as described above. In this example, the bone plate 300 does not comprise a relief in the second hole 208. FIGS. 6-9 show a simplified distal portion 204 of the bone plate 300 having a rectangular shape. It should be understood, however, that the bone plate 300 may be shaped substantially similarly to the bone plate 200. FIGS. 6-9 further include 5 dotted lines, e.g., a first line 302, a second line 304, a third line 306, a fourth line 308 and a fifth line 310, that remain in fixed position to illustrate the movement of the bone plate 300 relative to the bone as the bone fixation elements are inserted to apply the desired distal and cranial compressions.

FIG. 6 shows the distal portion 204 of the bone plate 300 according to one example with the first bone fixation element 212 inserted loosely into the bone via the first hole 206 and the second bone fixation element 214 inserted into the bone via the second hole 208 but not yet tightened to apply the desired compression. The first line 302 corresponds to an initial position of the proximal end of the simplified model of the distal portion 204 and the second line 304 corresponds to an initial position on the bone of a distal end of the distal portion 204. The third line 306 corresponds to a position at which the first bone fixation element 212 is inserted into the bone and the fourth line 308 corresponds to a position at which the second bone fixation element 214 is inserted into the bone. As would be understood, these lines do not move as compression is applied. Rather, the bone plate 300 moves over the stationary bone and relative to the stationary first and second bone fixation elements 212, 214 as compression is applied.

FIG. 7 shows the position of the bone plate 300 after application of the first compression. During this first compression, the bone plate 300 translates distally relative to the second bone fixation element 214. The bone plate 300 translates distally and cranially relative the first bone fixation element 212. The translation of the bone plate 300 relative to the first bone fixation element 212 rotates the bone plate 300 about the second bone fixation element 214 so that the distal end of the bone plate 300 rotates caudally relative to the second bone fixation element 214 and with the proximal end of the distal portion of the bone plate 300 rotating cranially relative to the second bone fixation element 214.

FIG. 8 shows the third bone fixation element 216 inserted into the bone via the third hole 210 of the bone plate 300 but not yet tightened to apply additional compression. The fifth line 310 corresponds to a position at which the third bone fixation element 216 is inserted into the bone via the third hole 210. FIG. 9 shows the bone plate 300 after the second compression has been applied. During this second compression due to the tightening of the third bone fixation element 216, the bone plate 300 translates distally relative to the third bone fixation element 216 and translates further distally and cranially relative the first bone fixation element 212. The translation of the bone plate 300 relative to the first bone fixation element 212 rotates the bone plate 300 about the third bone fixation element 216.

As seen in FIG. 9, during rotation of the bone plate 300 about the third bone fixation element 216, the second bone fixation element 214 is forced against the edge of the second hole 208 potentially interfering with the further movement of the bone plate 300 relative to the second bone fixation element 214 (i.e., potentially preventing the application of the full extent of the desired second compression). FIG. 10 shows the interference between the second bone fixation element 214 and the bone plate 200 during the second compression as it can be seen that, at the position of FIG. 9, the third bone fixation element 216 remains in the central portion of the third hole 210 (i.e., has not yet reached the proximal end of the third hole 210).

Referring back to the bone plate 200 described above, to accommodate the rotation of the bone plate 200 caused by the second compression, the relief portion 248 is cut into the second hole 208. FIG. 11 shows the detailed design of the second hole 208 of the bone plate 200 including the relief portion 248 configured to accommodate the distal/cranial plate shift while avoiding interference with the head of the second bone fixation element 214.

According to an exemplary method, the bone plate 200 is used to provide desired amounts of both cranial and distal compression during a TPLO procedure. As will be understood by those of skill in the art, a proximal segment of a tibia is cut away from a distal segment of the tibia, rotated and repositioned relative to the distal segment to a desired position and orientation relative to the stifle joint and the bone plate 200 is then placed over the separated bone segments to bind these bone segments together in a desired spatial relationship. The proximal portion 202 is positioned over the cut and repositioned proximal segment of the tibia in a desired alignment and the distal portion 204 is positioned over a target portion of the distal segment of the tibia and the bone plate 200 is coupled to the bone to maintain the desired spatial relationship between the segments of the tibia.

According to an exemplary method, the first bone fixation element 212 is inserted through the first hole 206 adjacent to the second end 238 and the second bone fixation element 214 is inserted through the second hole 208 adjacent to the second end 242 to establish a preliminary position of the bone plate 200 relative to the tibia so that the proximal portion 202 of the body 201 of the bone plate 200 is positioned over the cut proximal segment of the tibia and the distal portion 204 is positioned over the distal segment of the tibia. Once the preliminary position has been established, the bone fixation elements may be inserted through at least two of the holes extending through the proximal portion 202 of the bone plate 200 to fix the proximal portion 202 in a desired position relative to the proximal segment of the tibia. In one embodiment, bone fixation elements are inserted through a cranial one of the holes (e.g., third hole 232) and one of the other holes (e.g., the first hole 228 and the second hole 230) of the proximal portion 202.

The second bone fixation element 214 may then be tightened (e.g., rotatably inserted) further into the second hole 208 so that the head portion of the second bone fixation element 214 engages the compression surface 258 thereof, moving the bone plate 200 distally with respect to the second bone fixation element 214 to apply a first distal compression across the osteotomy cut—i.e., by moving the proximal segment of the tibia distally toward the distal segment of the tibia. As the bone plate 200 is being moved distally with respect the second bone fixation element 214 (i.e., the second bone fixation element 214 is being translated from the second end 242 toward the first end 240 of the second hole 208), the bone plate 200 translates relative to the first bone fixation element 212 along the axis B of the first hole 206, rotating the bone plate 200 about the second bone fixation element 214 and providing a simultaneous distal and cranial compression (i.e., the proximal portion 202 of the bone plate 200 is moved distally and in a cranial direction).

In particular, as the bone plate 200 moves so that the first bone fixation element 212 translates proximally along the axis B, the first bone fixation element 212 contacts a portion of the inner surface 250 forcing the proximal portion 202 of the bone plate 200 in the cranial direction.

As described above, the movement of the bone plate 200 generating the first distal compression moves the bone plate 200 relative to the first bone fixation element 212 so that the first bone fixation element 212 translates from its insertion position along the axis B to a central location, e.g., at or near the longitudinal axis L. The third bone fixation element 216 may then be inserted into the distal portion 246 so that the head portion of the third bone fixation element 216 engages the compression surface 258 thereof, moving the bone plate 200 further distally with respect to the distal segment of the tibia to apply a second distal compression across the osteotomy cut.

As the bone plate 200 is being moved distally with respect the third bone fixation element 216 (i.e., the bone plate 200 is translated so that the third bone fixation element 216 is translated from the distal portion 246 toward the proximal portion 244 of the third hole 210), the bone plate 200 translates so that the first bone fixation element 212 further translates along the axis B of the first hole 206, rotating the bone plate 200 about the third bone fixation element 216 and providing a second amount of simultaneous distal and cranial compression in which the proximal portion 202 of the bone plate 200 moves in a cranial direction. During the second distal compression the bone plate 200 rotates so that the second bone fixation element 214 is brought into the relief portion 248 of the second hole 208.

Upon completion of both cranial and distal compressions, the first bone fixation element 212 is tightened to fix the distal portion 204 of the bone plate 200 relative to the distal segment of the tibia. Additional bone fixation elements may be inserted through any remaining holes extending through to provide final fixation as desired. For example, bone fixation elements may be inserted through any remaining holes of the proximal portion 202, to provide further locking of the bone plate 200 in the desired position relative to the tibia.

As shown in FIG. 12, a bone plate 400 according to a further embodiment shows an alternate arrangement of the plate holes in the distal portion of the bone plate 400. The bone plate 400 comprises a body 401 including a proximal portion 402 sized and shaped to be positioned over and coupled to a first segment of bone and a distal portion 404 sized and shaped to extend along and be coupled to the second segment of bone. Those skilled in the art will understand that the proximal portion 402 may be substantially similar to the proximal portion 202 including holes configured to receive screws to couple the proximal portion 402 to the first segment 110 of the bone in any desired configuration. The distal portion 404 extends along a longitudinal axis L and includes a number of holes, e.g., three holes (408, 410 and 412), extending therethrough. The holes of the distal portion 404 are configured so that, when bone fixation elements are inserted in the holes, desired levels of distal (in the direction D) and cranial compression (in the direction C) of the osteotomy cut are provided at the interface between, e.g., the first and second segments 110, 112 of bone.

As indicated above, the amount of cranial compression depends on basic geometric relations between the angle of the axis b at which the angled hole 412 extends and the distances between 1) the screw about which the plate is rotating and a point (O) on the bone plate that is configured to overlie the osteotomy; and 2) the distance between the angled hole and the point O. Those skilled in the art will recognize that, in the bone plate 400 the angulation of the axis b is opposed to that of the axis B of the bone plate 200. This is because, in this case the hole 412 is distal of the holes 408 and 410. Thus, as the bone plate 400 is moved distally during distal compression applied via the holes 408, 410, the axis b is, relative to the point O, on the opposite side of the point of rotation of the bone plate 400 (e.g., a screw inserted in either the hole 408 or 410) and the distal portion of the bone plate 400 must move caudally to rotate the proximal portion 402 cranially. As indicated above, the desired amount of cranial compression will be achieved by selecting a total distal compression to be applied through the holes 408 and 410 and the location (relative to the point O) and angle of the axis b and the position (relative to the point O) of the holes (e.g., 408, 410) configured to receive the screw about which the bone plate 400 will rotate.

It will be understood by those of skill in the art that modifications and variations may be made in the structure and methodology of the present invention, without departing from the spirit and scope of the invention. Thus, it is intended that the present invention cover the modifications and variations of this invention, provided that they come within the scope of the appended claims and their equivalents.

What is claimed is:

1. A plate for providing cranial and distal compression of an osteotomy cut, comprising:
a body extending longitudinally from a proximal end to a distal end and defined via a first surface which, in an operative configuration, faces away from a bone and a second surface which, in the operative configuration, faces toward the bone, the body including a proximal portion configured to be positioned over a cut and repositioned proximal segment of the bone and a distal portion extending along a longitudinal axis L and configured to be positioned over a distal segment of the bone;
a first distal hole extending through a proximal end of the distal portion of the body from the first surface to the second surface, the first distal hole extending through the plate along an axis B angled relative to the axis L, the first distal hole being configured to slidably receive a first bone fixation element; and
a second distal hole extending through the distal portion of the body distally of the first distal hole from the first surface to the second surface, the second distal hole extending along the axis L and including a sloped compression surface along a distal portion thereof configured so that, when a second bone fixation element is inserted into the second distal hole, contact between a head of the second bone fixation element and the sloped compression surface of the second distal hole generates a first distal translation of the plate relative to the second bone fixation element, and wherein the first distal hole is configured such that, the first distal translation of the plate moves the plate relative to the first bone fixation element so that the first bone fixation element translates along the axis B toward a proximal end of the first distal hole, moving the proximal portion of the plate distally and cranially.

2. The plate of claim 1, wherein an angle between the axis L and the axis B along which the first distal hole extends corresponds to a desired cranial displacement.

3. The plate of claim 1, wherein the first distal hole is angled relative to the axis L so that a first end is proximal to a second end and the first end is separated transversely from the axis L on a caudal side of the axis L and the second end is separated transversely from the axis L on a cranial side of the axis L.

4. The plate of claim 3, wherein the translation of the first bone fixation element along the axis B caused by the first distal translation of the plate rotates the plate about the second bone fixation element so that the proximal portion of the plate is moved cranially.

5. The plate of claim 1, further comprising a third distal hole extending through the distal portion of the body, between the first and second distal holes, from the first surface to the second surface and extending along the axis L, a distal portion of the third distal hole including a sloped compression surface.

6. The plate of claim 5, wherein the third distal hole is configured as a combi-hole including a proximal portion configured as a locking hole and a distal portion configured as a dynamic compression hole, the proximal and distal portions of the combi-hole being open to and in communication with one another.

7. The plate of claim 6, wherein when the third distal hole is configured so that, as a third bone fixation element is inserted into the third distal hole the sloped compression surface of the third distal hole provides a second distal translation of the plate relative to the third bone fixation element, and wherein the first distal hole is configured such that, the second distal translation of the plate moves the plate relative to the first bone fixation element so that the plate moves relative to the first bone fixation element so that the first bone fixation element moves along the axis B toward a first end of the first distal hole, moving the proximal portion of the plate further distally and cranially.

8. The plate of claim 7, wherein the translation of the plate relative to the first bone fixation element along the axis B caused by the second distal translation of the plate causes the plate to rotate about the third bone fixation element so that the proximal portion of the plate moves cranially.

9. The plate of claim 8, wherein the plate is configured so that rotation of the plate about the third bone fixation element as the third bone fixation element is inserted into the third distal hole moves the second bone fixation element laterally relative to the axis L, and wherein the second distal hole comprises a relief portion sized, shaped and positioned to accommodate the lateral motion so that the second bone fixation element does not contact an inner surface of the second distal hole and prevent achievement of a desired degree of compression.

10. A method for providing cranial and distal compression of an osteotomy cut, comprising:
positioning a bone plate in a desired initial position with a first surface of the bone plate facing away from a tibia and a second surface thereof facing the tibia so that a distal portion of the bone plate extends over a distal tibial segment and a proximal portion of the bone plate extends over a proximal tibial segment that has been cut away from the proximal tibial segment, rotated and seated within a recess formed in the distal tibial segment when the proximal tibia segment was cut away;
coupling the proximal portion of the bone plate to the proximal tibial segment;
inserting a first distal bone fixation element into the distal tibial segment of the tibia via a first distal hole, the first distal hole extending along an axis B angled relative to a longitudinal axis L of a distal portion of the bone plate; and
inserting a second distal bone fixation element into the distal tibial segment via a second distal hole extending through the distal portion of the bone plate distally of the first distal hole so that a head portion of the second distal bone fixation element slides along a sloped compression surface forming an edge of a distal portion of the second distal hole to move the bone plate distally relative to the second distal bone fixation element pulling the proximal portion of the bone plate and the proximal tibial segment distally to provide a first distal compression between the proximal tibial segment and the distal tibial segment, the bone plate translating relative to the first distal bone fixation element so that the first distal bone fixation element translates proximally along the axis B during the first distal compression rotating the bone plate about the second distal bone fixation element so that the proximal portion of the bone plate moves cranially to provide a first cranial compression of the proximal tibial segment against the distal tibial segment.

11. The method of claim 10, wherein an angle between the axis L and the axis B corresponds to a desired cranial displacement and the first distal hole is angled so that a first end is proximal to a second end and the first end is separated transversely from the axis L on a caudal side of the axis L and the second end is separated transversely from the axis L on a cranial side of the axis L.

12. The method of claim 11, wherein the translation of the first distal bone fixation element along the axis B caused by the first distal translation of the bone plate rotates the bone plate about the second distal bone fixation element to move the proximal portion of the bone plate cranially.

13. The method of claim 12, wherein a third distal hole extends through the distal portion of the bone plate, between the first and second distal holes, from the first surface to the second surface, the third distal hole extending parallel to the axis L, a distal portion of the third distal hole including a sloped compression surface.

14. The method of claim 13, further comprising inserting a third bone fixation element into the third distal hole so that the sloped compression surface of the third distal hole provides a second distal translation of the bone plate relative to the third bone fixation element and a second cranial compression of the proximal tibial segment against the distal tibial segment.

15. The method of claim 14, wherein the rotation of the bone plate about the third bone fixation element moves the bone plate relative to the second distal bone fixation element so that the second distal bone fixation element moves laterally relative to the second distal hole, and wherein the second distal hole comprises a relief portion sized, shaped and positioned to accommodate the lateral motion of the second distal bone fixation element so that the second distal bone fixation element does not contact an inner surface of the second distal hole and prevent a desired degree of compression.
